(19) United States
(12) Patent Application Publication
(10) Pub. No.: US 2002/0058928 A1
(43) Pub. Date: May 16, 2002

(54) Intravenous tube coding system

(76) Inventor: Lani D. Antonio II, Hollister, CA (US)

Correspondence Address: HARSHAW RESEARCH INCORPORATED, P O BOX 418, OTTAWA, KS, 66067, US

(21) Appl. No.: 10/047,562

(22) Filed: Jan. 15, 2002

Related U.S. Application Data
(62) Division of application No. 09/348,082, filed on Jul. 6, 1999.

(51) Int. Cl. A61M 25/00

(52) U.S. Cl. 604/523; 604/189

(57) ABSTRACT

A system for the simultaneous intravenous administration of at least two different liquids to a patient that comprises a plurality of sleeves adapted to be removably mounted to a plurality of intravenous tubes with each sleeve having a corresponding but different identification member thereon for easily identifying and distinguishing the liquid flowing through each tube. Each identification member includes a different color indicium and pattern indicium that extend continuously along a substantial length of the corresponding sleeve. Each identification member may also include a shape indicium that presents a different cross-sectional configuration for identifying the fluid flowing through the corresponding intravenous tube.

### REFERENCE TO RELATED APPLICATIONS

This is a divisional (continuation) of co-pending application Ser. No. 09/348,082, filed Jul. 6, 1999.

### BACKGROUND OF THE INVENTION

This invention relates to the intravenous administration of medicaments and/or nutrients and, more particularly, to a system for coding intravenous tubes which allows efficient and accurate identification of fluids flowing through each tube.

It is a common medical practice to simultaneously deliver multiple drugs, nutrients, blood, and other fluids to a patient through multiple intravenous tubes. The ability to quickly and accurately identify which tube is delivering which fluid is extremely important when various medicaments are being delivered simultaneously, particularly in emergency or surgical situations. Obviously, disconnecting the wrong tube or mistakenly allowing incompatible drugs to be delivered simultaneously can lead to tragic results. The ability to distinguish various intravenous tubes even in the dark environment of an emergency response situation is also very important.

Various intravenous tube assemblies are known for identifying the various intravenous tubes delivering fluids to a patient. Some assemblies, for example those in U.S. Pat. Nos. 4,654,0265 and 423,750, disclose the singular use of color or numeric indicia to identify the fluid flowing through each tube. Although arguably effective in operation, these assemblies do not provide the most efficient means for quickly, easily, and accurately identifying the fluid flowing through a multitude of intravenous tubes, especially to those health care providers who are color blind or working in an otherwise darkened environment. Therefore, it is desirable to have a coding system for intravenous tubes in which the entire length of each tube allows a substantially unobstructed view of the fluid flowing therein. It is further desirable that the entire length of each tube includes identifying indicia. Further, it is desirable to have a coding system in which intravenous tubes can be distinguished by persons who are color blind or through touch only.

### SUMMARY OF THE INVENTION

In response thereto I have invented a system for the simultaneous intravenous administration of at least two different liquids to a patient which utilizes a plurality of transparent, colorless intravenous tubes. Each tube includes a corresponding but different means of identification thereon for easily and efficiently identifying a fluid flowing through each tube. Each identification means includes both a color indicia and a pattern indicia for the efficient identification of the fluid flowing through a particular tube. The color and pattern indicia extend longitudinally along the length of the corresponding intravenous tube. Each color indicia is different than any other color indicia and each pattern indicia is different from any other pattern indicia.

Each intravenous tube may also include a shape indicia in which each tube presents a cross-sectional configuration that is different than any other shape indicia. Each shape indicia also extends the length of the intravenous tube. Alternatively, each identification means may be on a plurality of elongated flexible sleeves which may be releasably attached to conventional intravenous tubes.

It is therefore a general object of this invention to provide an intravenous tube coding system for accurately administering a plurality of medicaments and/or nutrients to a medical patient.

Another object of this invention is to provide a coding system, as aforesaid, which allows quality control viewing of fluid administration along the entire length of each of a plurality of intravenous tubes.

Still another object of this invention is to provide a coding system, as aforesaid, in which indicia on intravenous tubes facilitates quick and accurate identification and distinction between tubes.

A further object of this invention is to provide a coding system, as aforesaid, which allows a plurality of intravenous tubes which can be identified by persons who are color blind or through touch only.

Other objects and advantages of this invention will become apparent from the following description taken in connection with the accompanying drawings, wherein is set forth by way of illustration and example, embodiments of this invention.

### BRIEF DESCRIPTION OF THE DRAWINGS

FIG. 1 is a side perspective view of the coding system showing portions of each intravenous tube, having a color coded indicia and a linear pattern indicia;

FIG. 2 is a side perspective view of an alternative embodiment of the coding system showing portions of each intravenous tube, having color coded indicia and an irregular pattern indicia;

FIG. 3 is a perspective view of another alternative embodiment of the coding system showing portions of each intravenous tube, having a color, pattern, and shape indicia;

FIG. 4 is a perspective view of another alternative embodiment of the coding system showing portions of intravenous tubes, each having a shape indicia with a different cross-sectional configuration;

FIG. 5 is a perspective view of another alternative embodiment of the coding system showing portions of each intravenous tube, having a shape indicia and a pattern indicia;

FIG. 6 is a perspective view of another embodiment of the coding system showing portions of each intravenous tube, having a shape indicia and a color indicia; and

FIG. 7 is a side view of another alternative embodiment of the coding system showing a portion of a sleeve, having a pattern indicia and color indicia thereon.

### DESCRIPTION OF THE PREFERRED EMBODIMENT

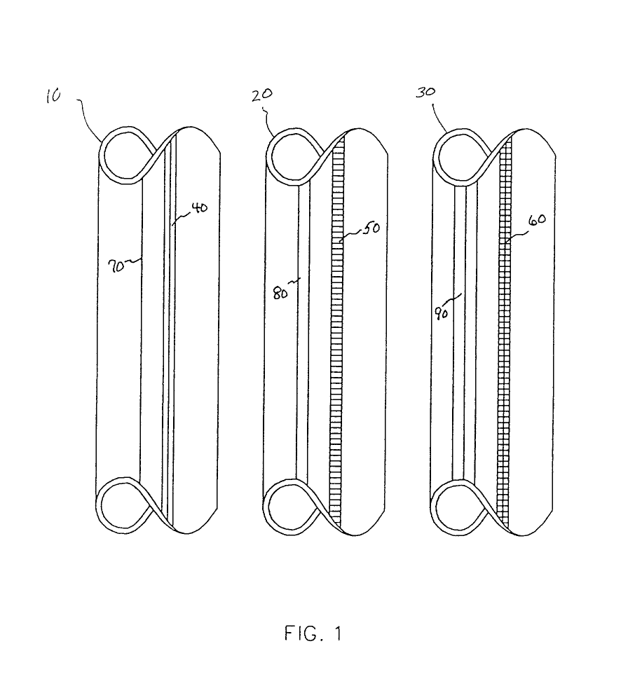

Turning more particularly to the drawings, FIG. 1 shows the preferred embodiment of the present invention having a first 10, second 20, and third 30 tube although any number of tubes may be utilized. Each tube 10, 20, 30 is formed of a flexible material that is transparent and colorless. Each tube 10, 20, 30 includes a narrow colored band or stripe 40, 50, 60 and a pattern indicium 70, 80, 90.

The colored bands 40, 50 ,60 extend longitudinally along the entire length of the tube. Alternatively, the narrow colored bands 40, 50, and 60 may extend in a spiral configuration along the length of each tube. The colored bands 40, 50, 60 are narrow enough to still allow a user to observe the fluid flow within a tube at any point therealong. Quality control of fluid administration, therefore, is conveniently monitored. The color of each band 40, 50, 60 on each tube is different. For example, as shown in FIG. 1, the first tube 10 includes a red band 40, the second tube 20 includes a blue band 50, and the third tube 30 includes a yellow band 60.

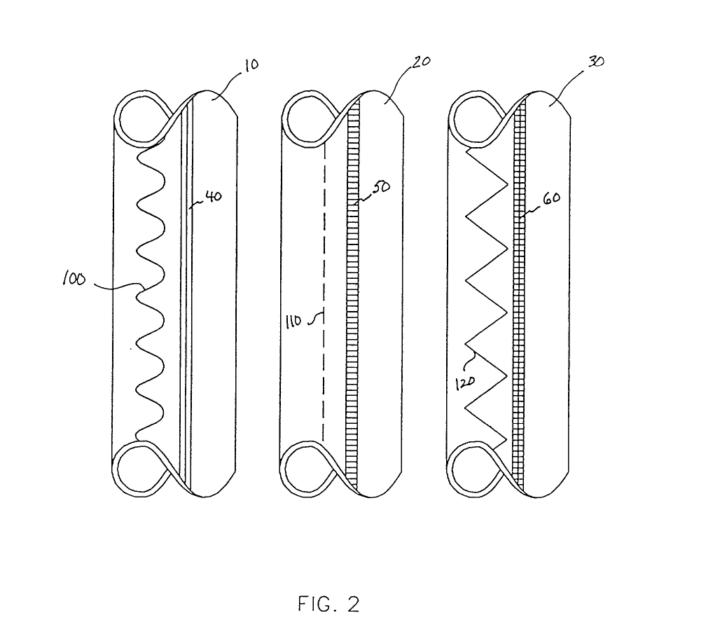

Each pattern indicium 70, 80, 90 is different from the other pattern indicia. The pattern indicia extend longitudinally along the entire length of each tube 10, 20, 30 (FIGS. 1 and 2) or may extend spirally thereabout (not shown). As shown, each pattern indicia include a different number of thin, spared apart straight lines 70, 80, 90 (FIG. 1) or, alternatively, irregular line patterns 100, 110, 120 (FIG. 2). Other more complex pattern configurations may also be used.

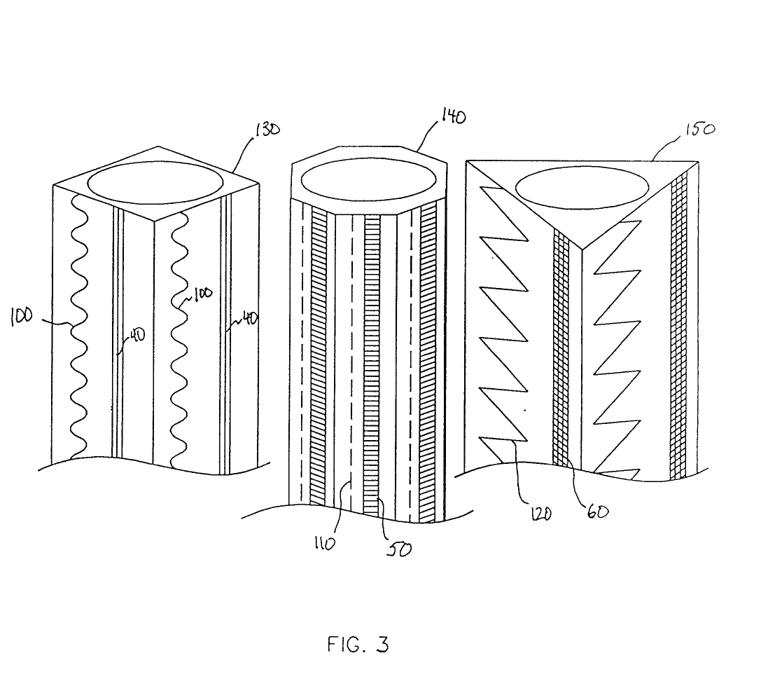

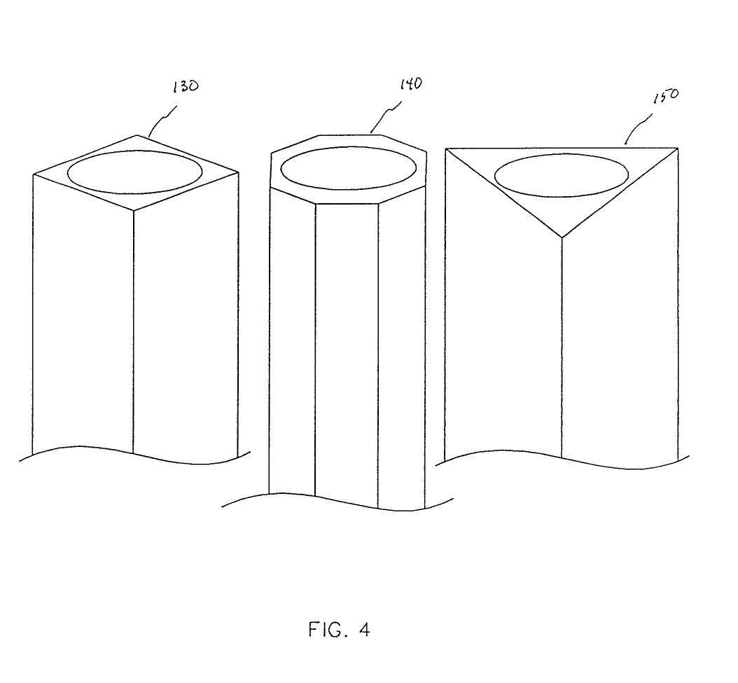

In addition to the color and pattern indicia, the intravenous tubes 10, 20, 30 may also include shape indicia for identifying the particular liquid flowing through a particular tube (FIG. 3). Each shape indicium 130, 140, 150 corresponds to a particular tube 10, 20, 30 and extends along the entire length thereof. Each shape indicium 130, 140, 150 presents a cross-sectional configuration that is different than any other of the shape indicia. The cross-section of each shape indicium is preferably configured according to a familiar geometric configuration that is readily identifiable by sight and touch, such as a square, octagon, triangle, etc. It is understood that the inner circumference of each intravenous tube is not modified by the shape indicia formed at each tube's exterior (FIG. 4).

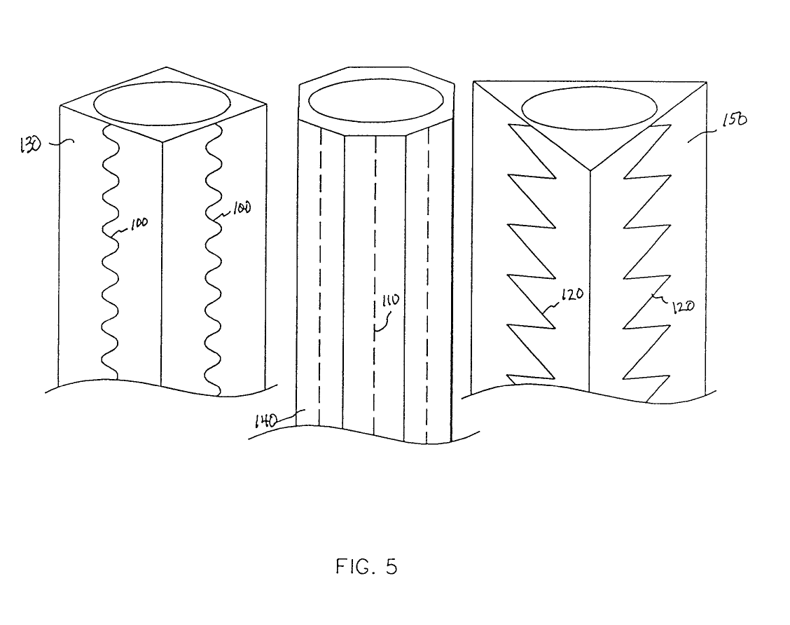

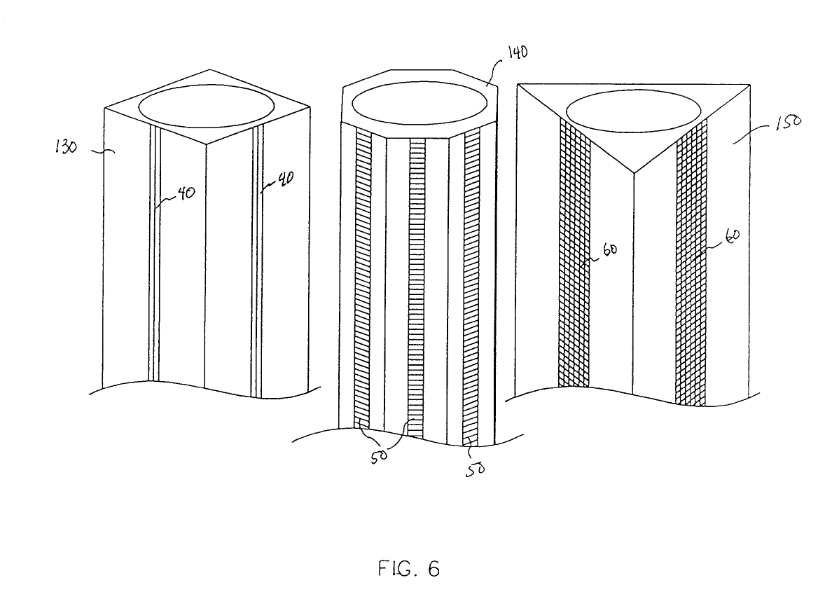

Alternatively, each intravenous tube 10, 20, 30 may include only a shape indicium 130, 140, 150 as in FIG. 4, each shape indicium 130, 140 or 150 being different from any other, as described previously. Further, each tube 10, 20, 30 may include both a shape indicium 130, 140, 150 and corresponding pattern indicium 100, 110, 120 as in FIG. 5 or a shape indicium and a corresponding color indicium 40, 50, 60 as in FIG. 6.

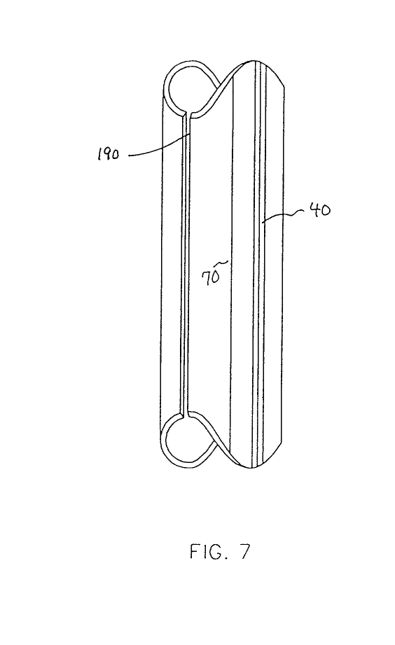

Instead of the tubes having the distinguishing indicia directly thereon, any of the indicia described previously may be included on a flexible sleeve 160 adapted for being securely mounted to a tube (FIG. 7). Each sleeve 160 includes a slit 190 along the longitudinal extent thereof such that a conventional intravenous tube may be inserted therein. The sleeves, such as sleeve 160, are preferably formed of resiliently flexible plastic for snugly snap-fitting onto a tube. Accordingly, sleeves are removable from tubes and may be reused for identifying liquids passing through multiple intravenous tubes of different patients.

It is understood that while certain forms of this invention have been illustrated and described, it is not limited thereto except insofar as such limitations are included in the following claims and allowable functional equivalents thereof.

Having thus described the invention, what is claimed as new and desired to be secured by Letters Patent is as follows:

1. A system for simultaneous intravenous administration of at least two different liquids to a patient through a plurality of intravenous tubes, said system comprising:
a plurality of elongate sleeves adapted to be removably mounted to intravenous tubes, said sleeves having identification means thereon for easily identifying a liquid flowing through said tubes; and
said identification means including a different color indicium extending continuously along a length of each said sleeve.

2. A system for the simultaneous intravenous administration of at least two different liquids to a patient as claimed in claim 1 wherein said identification means includes a different pattern indicium extending continuously along the length of each said sleeve.

3. A system for the simultaneous intravenous administration of at least two different liquids to a patient as claimed in claim 1 wherein said identification means includes a different shape indicium extending continuously along the length of each said sleeve.

4. A system for the simultaneous intravenous administration of at least two different liquids to a patient as claimed in claim 1 wherein each sleeve includes a slit along a longitudinal extent thereof and is formed of a resiliently flexible material for being mounted to a tube in a snap-fit relationship.

5. A system for simultaneous intravenous administration of at least two different liquids to a patient through a plurality of intravenous tubes, said system comprising:
a plurality of elongate sleeves, each sleeve being formed of a resiliently flexible material and having a slit along a longitudinal extent thereof for being selectively mounted to an intravenous tube in a snap-fit relationship, said sleeves having identification means thereon for easily identifying a liquid flowing through said tubes; and
said identification means including a different color indicium extending continuously along a length of each said sleeve.

6. A system for the simultaneous intravenous administration of at least two different liquids to a patient as claimed in claim 5 wherein said identification means includes a different pattern indicium extending continuously along the length of each said sleeve.

7. A system for the simultaneous intravenous administration of at least two different liquids to a patient as claimed in claim 6 wherein said identification means includes a different shape indicium extending continuously along the length of each said sleeve.

8. A system for the simultaneous intravenous administration of at least two different liquids to a patient as claimed in claim 5 wherein said identification means includes a different shape indicium extending continuously along the length of each said sleeve.

9. A system for simultaneous intravenous administration of at least two different liquids to a patient through a plurality of intravenous tubes, said system comprising:
a plurality of elongate sleeves, each sleeve being formed of a resiliently flexible material and having a slit along a longitudinal extent thereof for being selectively mounted to an intravenous tube in a snap-fit relationship, said sleeves having identification means thereon for easily identifying a liquid flowing through said tubes;
said identification means including a different color indicium extending continuously along a length of each said sleeve;
said identification means including a different pattern indicium extending continuously along the length of each said sleeve; and
said identification means including a different shape indicium extending continuously along the length of each said sleeve.
